(19) United States
(12) Patent Application Publication
(10) Pub. No.: US 2016/0317098 A1
(43) Pub. Date: Nov. 3, 2016

(54) IMAGING APPARATUS, IMAGE PROCESSING APPARATUS, AND IMAGE PROCESSING METHOD

(71) Applicant: OLYMPUS CORPORATION, Tokyo (JP)

(72) Inventor: Kazunori YOSHIZAKI, Tokyo (JP)

(73) Assignee: OLYMPUS CORPORATION, Tokyo (JP)

(21) Appl. No.: 14/977,396

(22) Filed: Dec. 21, 2015

Related U.S. Application Data
(63) Continuation of application No. PCT/JP2015/063048, filed on Apr. 30, 2015.

(51) Int. Cl. A61B 5/00; H04N 5/225; A61B 5/0205; A61B 1/06; A61B 1/00; H04N 5/33; A61B 1/04

(52) U.S. Cl. CPC A61B 5/7278; H04N 5/332; H04N 5/2256; A61B 1/04; A61B 1/0646; A61B 1/00009; A61B 1/0638; A61B 1/0661; A61B 5/0205; A61B 5/4884; A61B 5/443; A61B 5/489; A61B 5/0215

(57) ABSTRACT

An imaging apparatus includes: an imaging element that generates image data by photoelectrically converting light received by pixels; a filter array including a unit including different types of visible light filters with different transmission spectrum maximum values within a visible light band, and invisible light filters having a transmission spectrum maximum value in an invisible light range of wavelengths longer than those of the visible light band; a partial area detection unit that detects a partial area of the subject on an image corresponding to the image data; and a vital information generation unit that generates vital information on a subject based on image signals output by pixels, in an imaging area of the imaging element corresponding to the detected partial area, on which the invisible light filters are disposed.

### CROSS REFERENCES TO RELATED APPLICATIONS

This application is a continuation of International Application No. PCT/JP2015/063048, filed on Apr. 30, 2015, the entire contents of which are incorporated herein by reference.

### BACKGROUND OF THE INVENTION

1. Field of the Invention

The present invention relates to imaging apparatuses, image processing apparatuses, and image processing methods.

2. Description of the Related Art

In the medical field and the health field, as information to determine the state of human health, vital information such as a heart rate, oxygen saturation, and blood pressure has been used to determine the state of a subject's health. For example, there is a known technology that images, by an image sensor, a living body such as a finger brought into contact with the inside of a measurement probe that emits red light and near-infrared light, separately, and calculates the oxygen saturation of the living body, based on image data generated by the image sensor (see Japanese Laid-open Patent Publication No. 2013-118978). According to this technology, the oxygen saturation of a living body is calculated, based on the degree of light absorption by the living body calculated according to image data generated by the image sensor, and changes in the degree of light absorption over time.

### SUMMARY OF THE INVENTION

An imaging apparatus according to one aspect of the present invention generates image data for detecting vital information on a subject, and includes: an imaging element that generates the image data by photoelectrically converting light received by each of a plurality of pixels arranged two-dimensionally; a filter array including a unit including different types of visible light filters with different transmission spectrum maximum values within a visible light band, and invisible light filters having a transmission spectrum maximum value in an invisible light range of wavelengths longer than those of the visible light band, the visible light filters and the invisible light filters being disposed in correspondence with the plurality of pixels; a partial area detection unit that detects a partial area of the subject on an image corresponding to the image data generated by the imaging element; and a vital information generation unit that generates vital information on the subject, based on image signals output by pixels, in an imaging area of the imaging element corresponding to the partial area detected by the partial area detection unit, on which the invisible light filters are disposed.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a block diagram illustrating a functional configuration of an imaging apparatus according to a first embodiment of the present invention;

FIG. 2 is a diagram schematically illustrating a configuration of a filter array according to the first embodiment of the present invention;

FIG. 3 is a graph illustrating an example of the transmittance characteristics of each filter according to the first embodiment of the present invention;

FIG. 4 is a flowchart illustrating the outline of processing executed by the imaging apparatus according to the first embodiment of the present invention;

FIG. 5 is a diagram illustrating an example of an image corresponding to image data generated by the imaging apparatus according to the first embodiment of the present invention;

FIG. 6 is a graph schematically illustrating a heart rate as vital information generated by a vital information generation unit according to the first embodiment of the present invention;

FIG. 7 is a block diagram illustrating a functional configuration of an imaging apparatus according to a second embodiment of the present invention;

FIG. 8 is a diagram schematically illustrating a configuration of a filter array according to the second embodiment of the present invention;

FIG. 9 is a flowchart illustrating the outline of processing executed by the imaging apparatus according to the second embodiment of the present invention;

FIG. 10A is a diagram illustrating an example of an image corresponding to RGB data generated by the imaging apparatus according to the second embodiment of the present invention;

FIG. 10B is a diagram illustrating an example of an image corresponding to IR data generated by the imaging apparatus according to the second embodiment of the present invention;

FIG. 11 is a flowchart illustrating the outline of processing executed by an imaging apparatus according to a third embodiment of the present invention;

FIG. 12 is a diagram illustrating an example of an image corresponding to image data generated by the imaging apparatus according to the third embodiment of the present invention;

FIG. 13 is a diagram schematically illustrating partial areas generated by a vital information generation unit according to the third embodiment of the present invention;

FIG. 14 is a diagram schematically illustrating a plurality of partial areas detected by a partial area detection unit according to a first modification of the third embodiment of the present invention;

FIG. 15 is a graph schematically illustrating heart rates in each partial area illustrated in FIG. 14;

FIG. 16 is a diagram schematically illustrating a case where the vital information generation unit divides a partial area detected by the partial area detection unit into a plurality of areas to generate vital information according to a second modification of the third embodiment of the present invention;

FIG. 17 is a block diagram illustrating a functional configuration of an imaging apparatus according to a fourth embodiment of the present invention;

FIG. 18 is a graph illustrating the transmittance characteristics of an optical filter according to the fourth embodiment of the present invention;

FIG. 19 is a block diagram illustrating a functional configuration of an imaging apparatus according to a fifth embodiment of the present invention;

FIG. 20 is a graph illustrating the relationship between the transmittance characteristics of each filter and first wavelength light emitted by a first light source according to the fifth embodiment of the present invention;

FIG. 21 is a block diagram illustrating a functional configuration of an imaging apparatus according to a sixth embodiment of the present invention; and

FIG. 22 is a graph illustrating the relationship between the transmittance characteristics of an optical filter of the imaging apparatus and light of a first wavelength band emitted by a first light source and light of a second wavelength band emitted by a second light source according to the sixth embodiment of the present invention.

### DETAILED DESCRIPTION OF THE PREFERRED EMBODIMENTS

Hereinafter, embodiments to implement the present invention will be described in detail with the drawings. The embodiments below are not intended to limit the present invention. The drawings referred to in the description below only approximately illustrate shapes, sizes, and positional relationships to the extent that details of the present invention can be understood. That is, the present invention is not limited only to the shapes, sizes, and positional relationships illustrated in the drawings. The same components are denoted by the same reference numerals in the description.

### First Embodiment

### Configuration of Imaging Apparatus

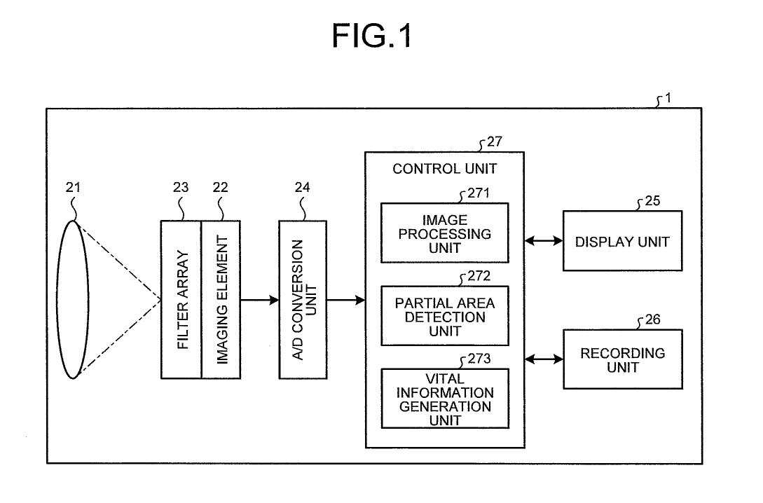

FIG. 1 is a block diagram illustrating a functional configuration of an imaging apparatus according to a first embodiment of the present invention. An imaging apparatus 1 illustrated in FIG. 1 includes an optical system 21, an imaging element 22, a filter array 23, an A/D conversion unit 24, a display unit 25, a recording unit 26, and a control unit (a controller or a processor) 27.

The optical system 21 is configured using one or a plurality of lenses such as a focus lens and a zoom lens, a diaphragm, and a shutter, or the like, to form a subject image on a light-receiving surface of the imaging element 22.

The imaging element 22 receives light of a subject image that has passed through the filter array 23, and photoelectrically converts it, thereby generating image data continuously according to a predetermined frame (60 fps). The imaging element 22 is configured using a complementary metal oxide semiconductor (CMOS), a charge coupled device (COD), or the like, which photoelectrically converts light that has passed through the filter array 23 and received by each of a plurality of pixels arranged two-dimensionally, and generates electrical signals.

The filter array 23 is disposed on the light-receiving surface of the imaging element 22. The filter array 23 has a unit including a plurality of visible light filters with different transmission spectrum maximum values within a visible light band, and invisible light filters having a transmission spectrum maximum value in an invisible light range of wavelengths longer than those of a visible light range, disposed in correspondence with the plurality of pixels in the imaging element 22.

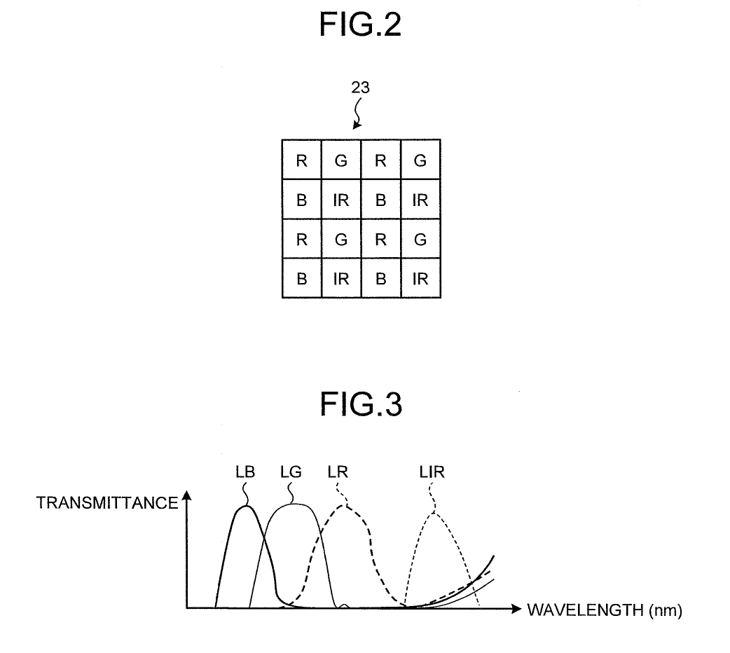

FIG. 2 is a diagram schematically illustrating a configuration of the filter array 23. As illustrated in FIG. 2, the filter array 23 is disposed on respective light-receiving surfaces of the pixels constituting the imaging element 22, and has a unit including visible light filters R that transmit red light, visible light filters G that transit green light, visible light filters B that transmit blue light, and invisible light filters IR that transmit invisible light, disposed in correspondence with the plurality of pixels. Hereinafter, a pixel on which a visible light filter R is disposed is described as an R pixel, a pixel on which a visible light filter G is disposed as a G pixel, a pixel on which a visible light filter B is disposed as a B pixel, and a pixel on which an invisible light filter IR is disposed as an IR pixel. An image signal output by an R pixel is described as R data, an image signal output by a G pixel as G data, an image signal output by a B pixel as B data, and an image signal output by an IR pixel as IR data.

FIG. 3 is a graph illustrating an example of the transmittance characteristics of each filter. In FIG. 3, the horizontal axis represents wavelength (nm), and the vertical axis represents transmittance. In FIG. 3, a curved line LR represents the transmittance of the visible light filters R, a curved line LG represents the transmittance of the visible light filters G, a curved line LB represents the transmittance of the visible light filters B, and a curved line LIR represents the transmittance of the invisible light filters IR. In FIG. 3, although the transmittance characteristics of each filter are illustrated to simplify the description, they are equal to the spectral sensitivity characteristics of each pixel (R pixels, G pixels, B pixels, and IR pixels) when each pixel is provided with a respective filter.

As illustrated in FIG. 3, the visible light filters R have a transmission spectrum maximum value in a visible light band. Specifically, the visible light filters R have the transmission spectrum maximum value in a wavelength band of 620 to 750 nm, and transmit light of the wavelength band of 620 to 750 nm, and also transmit part of light of a wavelength band of 850 to 950 nm in an invisible light range. The visible light filters G have a transmission spectrum maximum value in the visible light band. Specifically, the visible light filters G have the transmission spectrum maximum value in a wavelength band of 495 to 570 nm, and transmit light of the wavelength band of 495 to 570 nm, and also transmit part of light of the wavelength band of 850 to 950 nm in the invisible light range. The visible light filters B have a transmission spectrum maximum value in the visible light band. Specifically, the visible light filters B have the transmission spectrum maximum value in a wavelength band of 450 to 495 nm, and transmit light of the wavelength band of 450 to 495 nm, and also transmit part of light of the wavelength band of 850 to 950 nm in the invisible light range. The invisible light filters IR have a transmission spectrum maximum value in an invisible light band, and transmit light of the wavelength band of 850 to 950 nm.

Returning to FIG. 1, the description of the configuration of the imaging apparatus 1 will be continued.

The A/D conversion unit 24 converts analog image data input from the imaging element 22 to digital image data, and outputs it to the control unit 27.

The display unit 25 displays images corresponding to image data input from the control unit 27. The display unit 25 is configured using a liquid crystal or organic electro luminescence (EL) display panel, or the like.

The recording unit 26 records various kinds of information on the imaging apparatus 1. The recording unit 26 records image data generated by the imaging element 22, various programs for the imaging apparatus 1, parameters for processing being executed, and the like. The recording unit 26 is configured using synchronous dynamic random access memory (SDRAM), flash memory, a recording medium, or the like.

The control unit 27 performs instructions, data transfer, and so on to units constituting the imaging apparatus 1, thereby centrally controlling the operation of the imaging apparatus 1. The control unit 27 is configured using a central processing unit (CPU), a processor or the like. In the first embodiment, the control unit 27 functions as an image processing apparatus.

Here, a detailed configuration of the control unit 27 will be described. The control unit 27 includes at least an image processing unit (an image processor) 271, a partial area detection unit 272, and a vital information generation unit 273.

The image processing unit 271 performs predetermined image processing on image data input from the A/D conversion unit 24. Here, the predetermined image processing includes optical black subtraction processing, white balance adjustment processing, image data synchronization processing, color matrix arithmetic processing, γ correction processing, color reproduction processing, and edge enhancement processing. Further, the image processing unit 271 performs demosaicing processing using R data, G data, and B data output by the R pixels, the G pixels, and the B pixels, respectively. Specifically, without using IR data output by the IR pixels, the image processing unit 271 performs the demosaicing processing by interpolating IR data of the IR pixels with data output by other pixels (R pixels, G pixels, or B pixels).

The partial area detection unit 272 detects a predetermined partial area on an image corresponding to RGB data in image data input from the A/D conversion unit 24. Specifically, the partial area detection unit 272 performs pattern matching processing on an image, thereby detecting an area containing the face of a subject. Other than the face of a subject, the partial area detection unit 272 may detect a skin area of a subject, based on color components included in an image.

The vital information generation unit 273 generates vital information on a subject, based on IR data output by IR pixels of pixels in an imaging area of the imaging element 22 corresponding to a partial area detected by the partial area detection unit 272 (hereinafter, referred to as “IR data on a partial area”). Here, the vital information is at least one of blood pressure, a heart rate, heart rate variability, stress, oxygen saturation, skin moisture, and a vein pattern.

The imaging apparatus 1 configured like this images a subject, thereby generating image data used for detecting vital information on the subject.

Processing by Imaging Apparatus

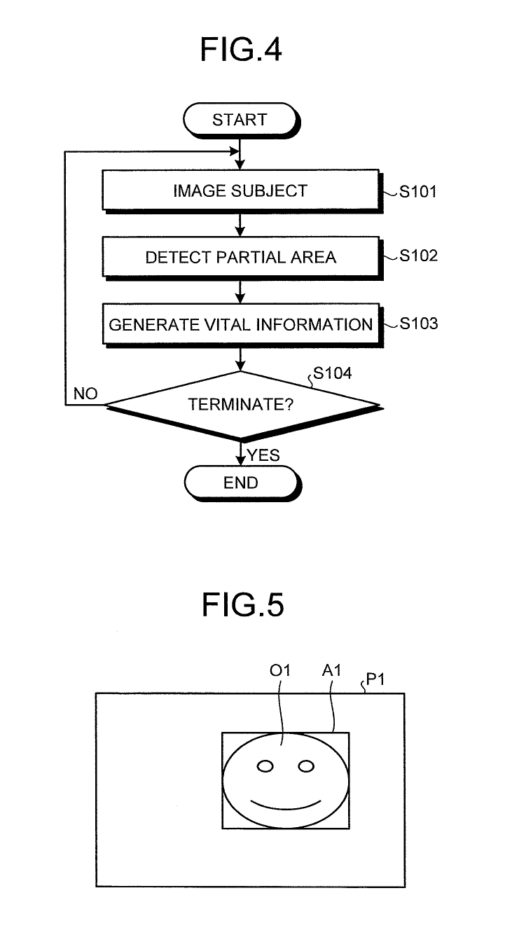

Next, processing executed by the imaging apparatus 1 will be described. FIG. 4 is a flowchart illustrating the outline of the processing executed by the imaging apparatus 1.

As illustrated in FIG. 4, first, the imaging element 22 continuously images a subject according to the predetermined frame rate, sequentially generating temporally-continuous image data (step S101).

Next, the partial area detection unit 272 detects a partial area of the subject on an image corresponding to RGB data in the image data generated by the imaging element 22 (step S102). Specifically, as illustrated in FIG. 5, the partial area detection unit 272 detects a partial area A1 containing the face of a subject O1 using pattern matching technology, on an image P1 corresponding to RGB data in the image data generated by the imaging element 22.

Thereafter, the vital information generation unit 273 generates vital information on the subject, based on IR data on the partial area detected by the partial area detection unit 272 (step S103). Specifically, the vital information generation unit 273 explains the heart rate of the subject as vital information, based on IR data by the partial area detection unit 272.

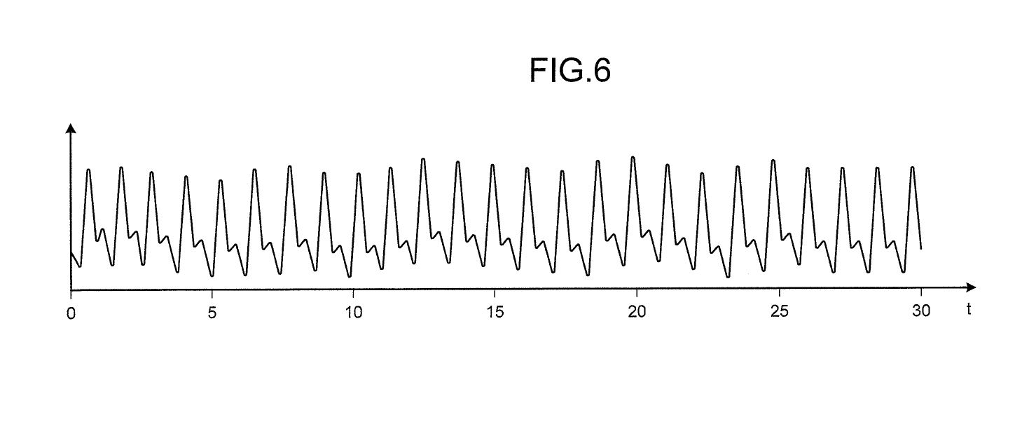

FIG. 6 is a graph schematically illustrating a heart rate as vital information generated by the vital information generation unit 273. In FIG. 6, the horizontal axis represents time, and the vertical axis represents the mean value of IR data on a partial area.

As illustrated in FIG. 6, the vital information generation unit 273 calculates mean values of IR data on the partial area detected by the partial area detection unit 272, and calculates the heart rate of the subject by counting the number of maximum values of the mean values, thereby generating vital information.

Returning to FIG. 4, the description of step S104 and thereafter will be continued.

In step S104, when generation of vital information on the subject is terminated (step S104: Yes), the imaging apparatus 1 ends the processing. On the other hand, when generation of vital information on the subject is not terminated (step S104: No), the imaging apparatus 1 returns to step S101.

According to the first embodiment of the present invention described above, vital information on a living body can be obtained even in a state of not contacting the living body because the vital information generation unit 273 generates vital information on a subject, based on IR data on a partial area detected by the partial area detection unit 272.

Further, according to the first embodiment of the present invention, accuracy in obtaining vital information can be improved because the vital information generation unit 273 generates vital information on a subject, based on image signals output by pixels on which invisible light filters are disposed, of pixels in an imaging area of the imaging element 22 corresponding to a partial area detected by the partial area detection unit 272.

Furthermore, according to the first embodiment of the present invention, high-accuracy vital information can be generated from moving image data because the partial area detection unit 272 sequentially detects a partial area every time image data is generated by the imaging element 22, and the vital information generation unit 273 generates vital information every time a partial area is detected by the partial area detection unit 272.

Moreover, according to the first embodiment of the present invention, vital information processing time can be speeded up by the fact that image processing such as demosaicing processing can be omitted because the vital information generation unit 273 generates vital information using IR data (RAW data) output from IR pixels.

### Second Embodiment

Next, a second embodiment of the present invention will be described. An imaging apparatus according to the second embodiment is different in the configuration of the filter array 23 of the imaging apparatus 1 according to the above-described first embodiment, and also different in the detection method in which the partial area detection unit 272 detects a partial area. Thus, hereinafter, after a configuration of a filter array of the imaging apparatus according to the second embodiment is described, processing executed by the imaging apparatus according to the second embodiment will be described.

Configuration of Imaging Apparatus

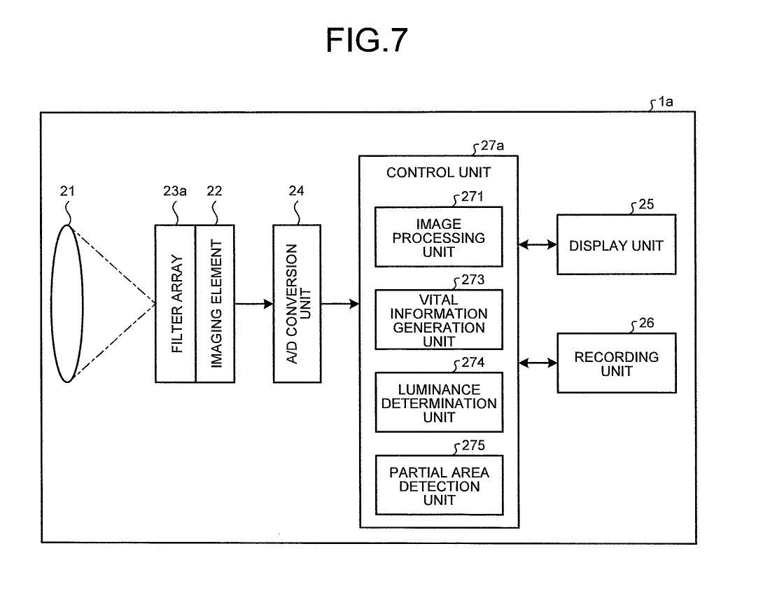

FIG. 7 is a block diagram illustrating a functional configuration of an imaging apparatus according to the second embodiment of the present invention. An imaging apparatus 1a illustrated in FIG. 7 includes a filter array 23a and a control unit 27a in place of the filter array 23 and the control unit 27 of the imaging apparatus 1 according to the above-described first embodiment, respectively.

The filter array 23a forms a predetermined array pattern using a plurality of visible light filters with different transmission spectrum maximum values in a visible light band, and a plurality of invisible light filters with different transmission spectrum maximum values in different invisible light ranges, invisible light ranges of wavelengths longer than those of a visible light range. The filters forming the array pattern are each disposed in a position corresponding to one of the plurality of pixels of the imaging element 22.

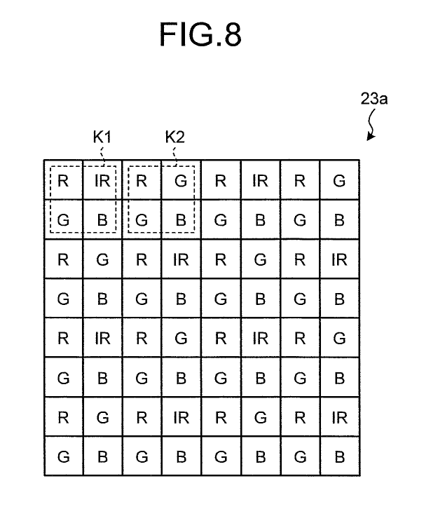

FIG. 8 is a diagram schematically illustrating a configuration of the filter array 23a. As illustrated in FIG. 8, the filter array 23a is formed by a pattern of repetitions of a set (4×4) consisting of two array units each of which is a set K1 of a visible light filter R, a visible light filter G, a visible light filter B, and an invisible light filter IR, and two Bayer array units each of which is a set K2 of a visible light filter R, two visible light filters G, and a visible light filter B. In the filter array 23a, the number of the invisible light filters IR is lower than the number of the visible light filters R, the number of the visible light filters G, and the number of the visible light filters B (R>IR, G>IR, B>IR).

The control unit 27a performs instructions, data transfer, and so on to units constituting the imaging apparatus 1a, thereby centrally controlling the operation of the imaging apparatus 1a. The control unit 27a includes an image processing unit 271, a partial area detection unit 275, a vital information generation unit 273, and a luminance determination unit 274. In the second embodiment, the control unit 27a serves as an image processing apparatus.

The luminance determination unit 274 determines whether or not an image corresponding to image data input from the A/D conversion unit 24 has predetermined luminance or more. Specifically, the luminance determination unit 274 determines whether or not RGB image data included in image data exceeds a predetermined value.

When the luminance determination unit 274 determines that an image corresponding to image data input from the A/D conversion unit 24 has the predetermined luminance or more, the partial area detection unit 275 performs pattern matching processing on an image corresponding to RGB data, thereby detecting a partial area containing the face or skin of a subject. On the other hand, when the luminance determination unit 274 determines that an image corresponding to image data input from the A/D conversion unit 24 does not have the predetermined luminance or more, the partial area detection unit 275 performs the pattern matching processing on an image corresponding to RGB data and IR data, thereby detecting a partial area containing the face or skin of a subject.

Processing by Imaging Apparatus

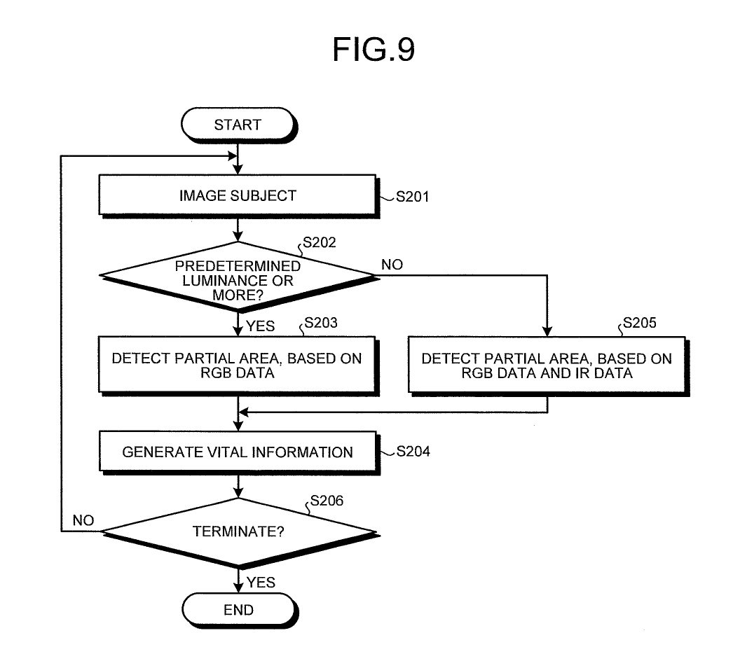

Next, processing executed by the imaging apparatus 1a will be described. FIG. 9 is a flowchart illustrating the outline of the processing executed by the imaging apparatus 1a.

As illustrated in FIG. 9, first, the imaging element 22 continuously images a subject, sequentially generating temporally-continuous image data (step S201).

Next, the luminance determination unit 274 determines whether or not an image corresponding to the image data input from the A/D conversion unit 24 has the predetermined luminance or more (step S202). When the luminance determination unit 274 determines that the image corresponding to the image data input from the A/D conversion unit 24 has the predetermined luminance or more (step S202: Yes), the imaging apparatus 1a proceeds to step S203 described below. On the other hand, when the luminance determination unit 274 determines that the image corresponding to the image data input from the A/D conversion unit 24 does not have the predetermined luminance or more (step S202: No), the imaging apparatus 1a proceeds to step S205 described below.

In step S203, the partial area detection unit 275 performs the pattern matching processing on an image corresponding to RGB data, thereby detecting a partial area containing the face or skin of the subject.

Next, the vital information generation unit 273 generates vital information on the subject, based on IR data on the partial area detected by the partial area detection unit 275 (step S204). After step S204, the imaging apparatus 1a proceeds to step S206 described below.

In step S205, the partial area detection unit 275 performs the pattern matching processing on an image corresponding to RGB data and IR data, thereby detecting a partial area containing the face or skin of the subject. After step S205, the imaging apparatus 1a proceeds to step S206 described below.

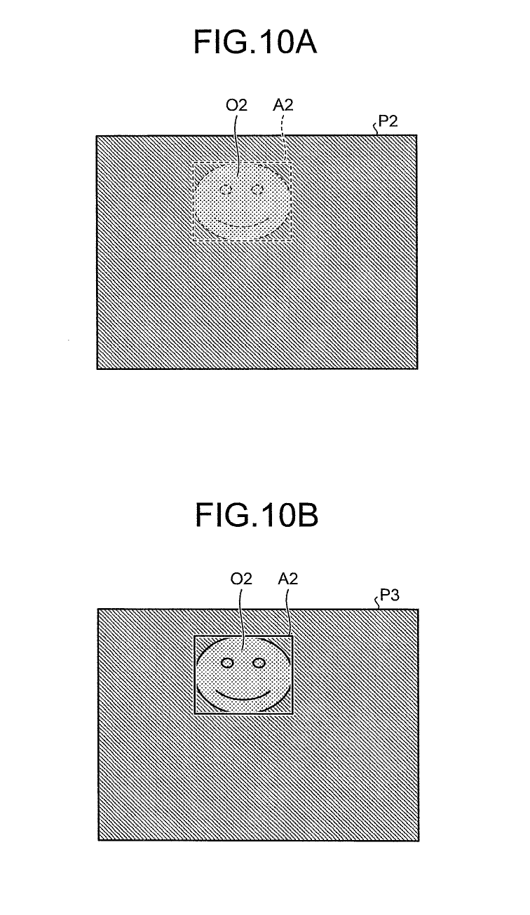

FIG. 10A is a diagram illustrating an example of an image corresponding to RGB data. FIG. 10B is a diagram illustrating an example of an image corresponding to RGB data and IR data. FIGS. 10A and 10B illustrate images when the imaging apparatus 1a images a subject in a dark place. As illustrated in FIGS. 10A and 10B, when a surrounding environment of a subject O2 is dark, it is usually difficult for the partial area detection unit 275 to detect a partial area A2 containing the face of the subject O2 only with a normal image P2 corresponding to RGB data because respective signal values of R pixels, G pixels, and B pixels are low (luminance is low). Therefore, in the second embodiment, the partial area detection unit 275 further uses IR data output by IR pixels in addition to RGB data, thereby detecting the partial area A2 containing the face of the subject O2. That is, in the second embodiment, as illustrated in FIG. 10B, when the luminance determination unit 274 determines that the image corresponding to the image data input from the A/D conversion unit 24 does not have the predetermined luminance or more, the partial area detection unit 275 performs the pattern matching processing on an image P3 corresponding to the RGB data and the IR data. This allows the detection of the partial area A2 containing the face or skin of the subject even when the imaging area is dark.

In step S206, when generation of vital information on the subject is terminated (step S206: Yes), the imaging apparatus 1a ends the processing. On the other hand, when generation of vital information on the subject is not terminated (step S206: No), the imaging apparatus 1a returns to step S201.

According to the second embodiment of the present invention described above, accuracy in obtaining vital information can be improved because the vital information generation unit 273 generates vital information on a subject, based on image signals output from pixels on which invisible light filters and visible light filters are disposed in an imaging area of the imaging element 22 corresponding to a partial area detected by the partial area detection unit 275.

Further, according to the second embodiment of the present invention, high-precision normal images (high resolution) can be obtained because the number of invisible light filters is lower than the number of a plurality of visible light filters of each type.

Furthermore, according to the second embodiment of the present invention, even when an imaging area is dark, a partial area containing the face or skin of a subject can be detected precisely because when the luminance determination unit 274 determines that an image corresponding to RGB data does not have the predetermined luminance or more, the partial area detection unit 275 performs the pattern matching processing on an image corresponding to the RGB data and IR data, thereby detecting a partial area containing the face or skin of the subject.

### Third Embodiment

Next, a third embodiment of the present invention will be described. An imaging apparatus according to the third embodiment has the same configuration as that of the imaging apparatus 1 according to the above-described first embodiment, and is different in processing executed. Specifically, in the imaging apparatus 1 according to the above-described first embodiment, the partial area detection unit 272 detects only a single partial area, but a partial area detection unit of the imaging apparatus according to the third embodiment detects a plurality of partial areas. Thus, hereinafter, only processing executed by the imaging apparatus according to the third embodiment will be described. The same components as those of the imaging apparatus 1 according to the above-described first embodiment are denoted by the same reference numerals and will not be described.

Processing by Imaging Apparatus

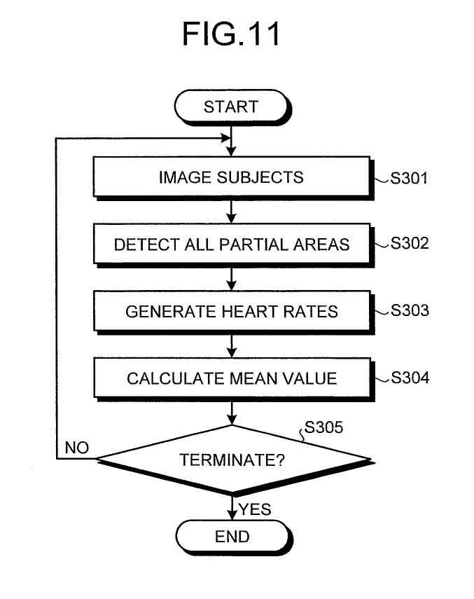

FIG. 11 is a flowchart illustrating the outline of processing executed by an imaging apparatus 1 according to the third embodiment of the present invention.

As illustrated in FIG. 11, first, an imaging element 22 images subjects and generates image data (step S301).

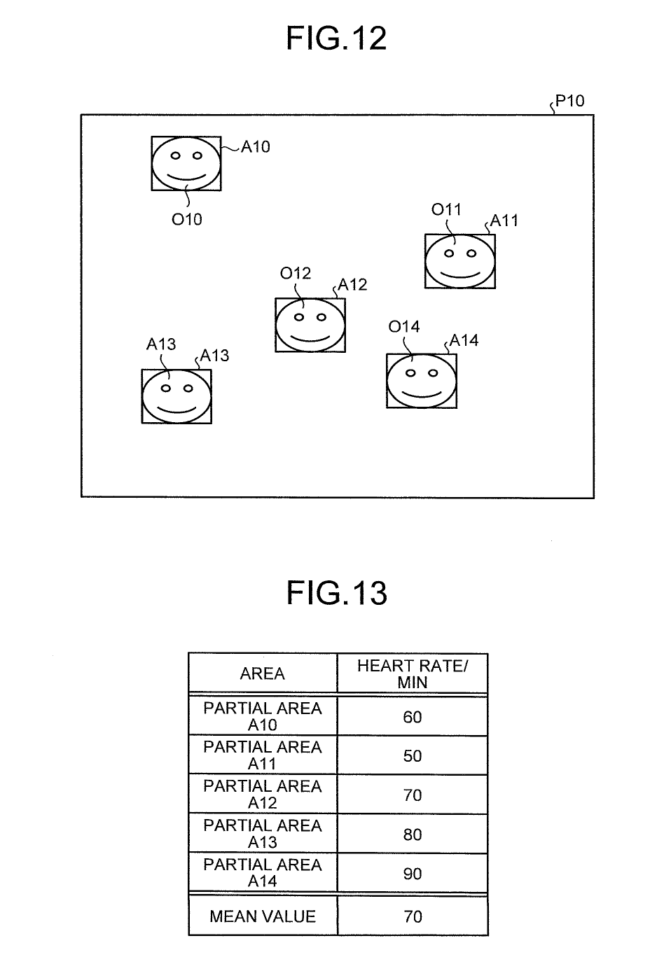

Next, a partial area detection unit 272 performs pattern matching processing on an image corresponding to the image data generated by the imaging element 22, thereby detecting partial areas of all the subjects included in the image (step S302). Specifically, as illustrated in FIG. 12, the partial area detection unit 272 performs the pattern matching processing on an image P10 corresponding to the image data generated by the imaging element 22, thereby detecting areas containing the faces of all subjects O10 to O14 included in the image P10 as partial areas A10 to A14.

Thereafter, a vital information generation unit 273 generates heart rates as respective vital information on the subjects O10 to O14, based on respective IR data on the plurality of partial areas detected by the partial area detection unit 272 (step S303). Specifically, as illustrated in FIG. 13, the vital information generation unit 273 generates heart rates as respective vital information on the subjects O10 to O14, based on respective IR data on the plurality of partial areas detected by the partial area detection unit 272.

Thereafter, as illustrated in FIG. 13, the vital information generation unit 273 calculates the mean value of the respective heart rates in the plurality of partial areas detected by the partial area detection unit 272 (step S304). This allows generation of a state of mass psychology as vital information. Although the vital information generation unit 273 calculates the mean value of the respective heart rates in the plurality of partial areas detected by the partial area detection unit 272, it may perform weighting for each of the plurality of partial areas detected by the partial area detection unit 272. For example, the vital information generation unit 273 may perform weighting for heart rates according to sex, age, face areas, or the like.

Next, when generation of vital information is terminated (step S305: Yes), the imaging apparatus 1 ends the processing. On the other hand, when generation of vital information is not terminated (step S305: No), the imaging apparatus 1 returns to step S301.

According to the third embodiment of the present invention described above, for example, a state of mass psychology can be generated as vital information because the partial area detection unit 272 performs the pattern matching processing on an image corresponding to image data generated by the imaging element 22, thereby detecting partial areas of all subjects included in the image.

### First Modification of Third Embodiment

Although the partial area detection unit 272 detects the faces of a plurality of subjects in the third embodiment of the present invention, it may detect a plurality of partial areas on a single person.

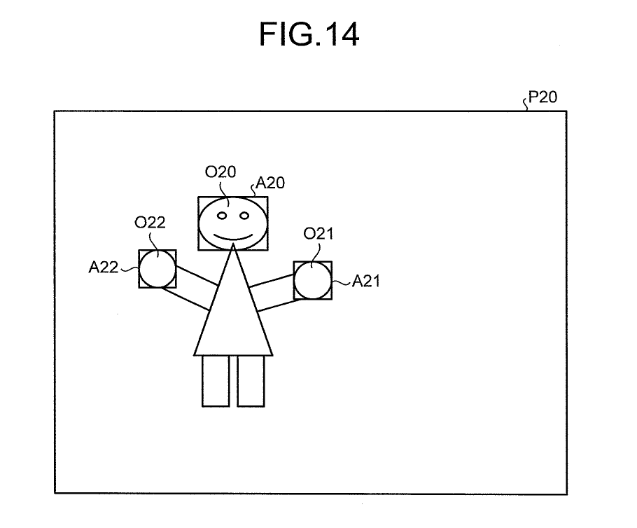

FIG. 14 is a diagram schematically illustrating a plurality of partial areas detected by the partial area detection unit 272. As illustrated in FIG. 14, the partial area detection unit 272 detects an area containing the face of a subject O20, and areas O21 and O22 containing the hands (skin color) of the subject O20 in an image P20 corresponding to RGB data generated by the imaging element 22, as partial areas A20 to A22, respectively.

Next, the vital information generation unit 273 generates heart rates of the subject O20 as vital information, based on IR data on the partial areas A20 to A22 detected by the partial area detection unit 272. Thereafter, the vital information generation unit 273 generates the degree of arteriosclerosis in the subject O20 as vital information.

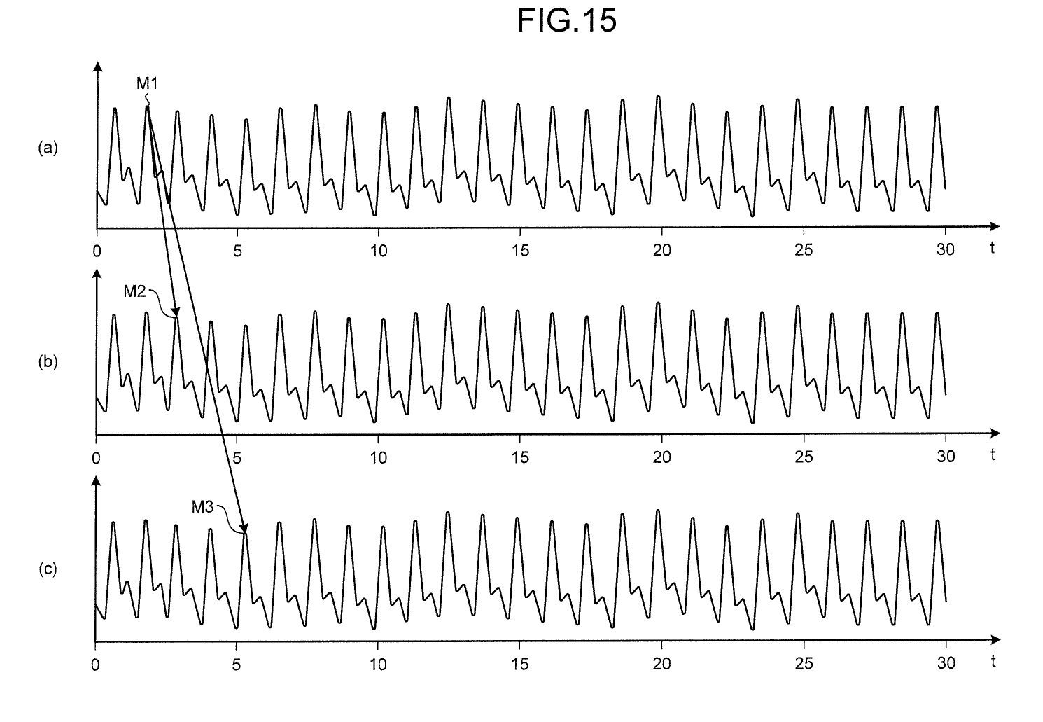

FIG. 15 is a graph schematically illustrating heart rates in the partial areas illustrated in FIG. 14. In FIG. 15, the horizontal axis represents time. In FIG. 15, FIG. 15(a) illustrates a heart rate in the above-described partial area A20, FIG. 15(b) illustrates a heart rate in the above-described partial area A21, and FIG. 15(c) illustrates a heart rate in the above-described partial area S22.

The vital information generation unit 273 generates the degree of arteriosclerosis in the subject O20 as vital information, based on the amount of difference between maximum values of respective heartbeats in the partial areas A20 to A22. Specifically, as illustrated in FIG. 15, it generates the degree of arteriosclerosis in the subject O20 as vital information, based on the amount of difference (phase difference) between maximum values M1 to M3 of respective heartbeats in the partial areas A20 to A22.

According to the first modification of the third embodiment of the present invention described above, arteriosclerosis in a subject can be determined because the partial area detection unit 272 detects a plurality of partial areas on the same subject, and the vital information generation unit 273 generates heart rates at a plurality of areas of the subject using IR data on the plurality of partial areas of the same subject detected by the partial area detection unit 272.

### Second Modification of Third Embodiment

In a second modification of the third embodiment of the present invention, the vital information generation unit 273 may divide a partial area containing the face of a subject detected by the partial area detection unit 272 into a plurality of areas, and generate vital information on each area.

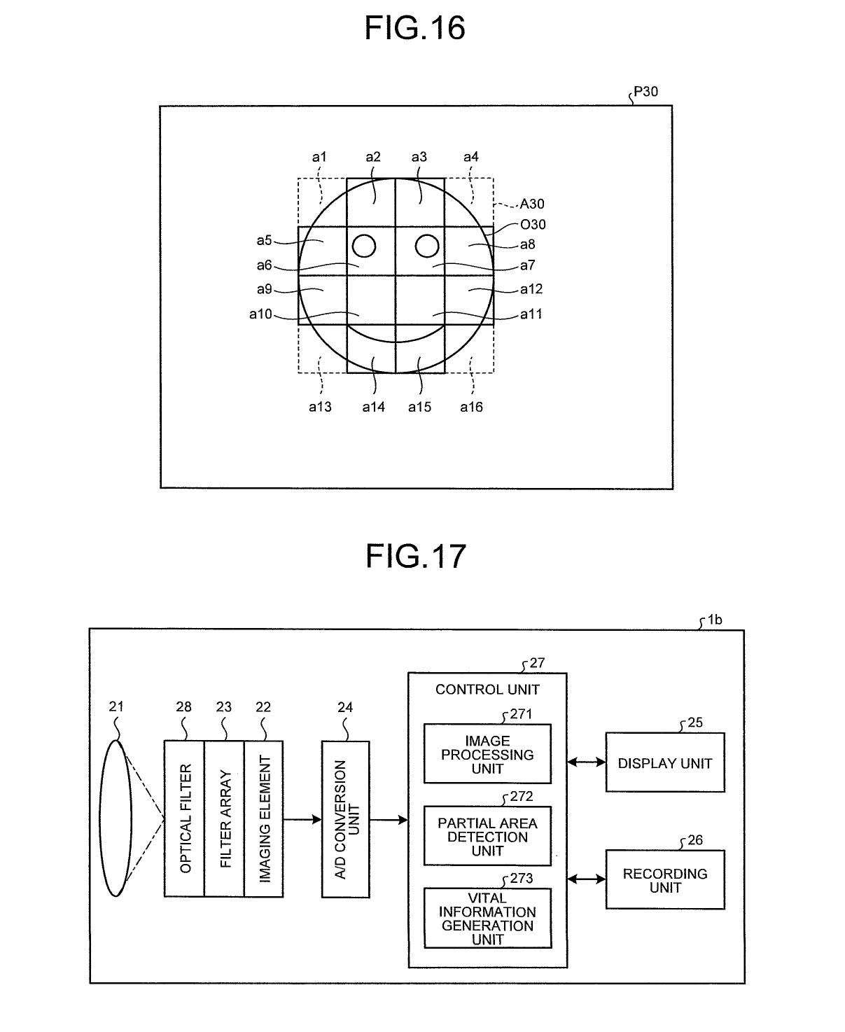

FIG. 16 is a diagram schematically illustrating a case where the vital information generation unit 273 divides a partial area detected by the partial area detection unit 272 into a plurality of areas to generate vital information. As illustrated in FIG. 16, the vital information generation unit 273 divides a partial area A30 containing the face of a subject O30 in an image P30 corresponding to RGB data generated by the imaging element 22, detected by the partial area detection unit 272 into a plurality of areas a1 to a16 (4×4), and generates vital information on the plurality of areas a1 to a16, based on respective IR data on the plurality of divided areas a1 to a16. In this case, the vital information generation unit 273 generates vital information by excluding areas a1, a4, a13, and a16 in the four corners.

According to the second modification of the third embodiment of the present invention described above, higher-accuracy vital information can be obtained because the vital information generation unit 273 divides a partial area detected by the partial area detection unit 272 into a plurality of areas, and generates vital information on the plurality of areas.

### Fourth Embodiment

Next, a fourth embodiment of the present invention will be described. An imaging apparatus according to the fourth embodiment is different in configuration from the imaging apparatus 1 according to the above-described first embodiment. Specifically, in the imaging apparatus according to the fourth embodiment, an optical filter that transmits only light of a predetermined wavelength band is disposed between an optical system and a filter array. Thus, hereinafter, a configuration of the imaging apparatus according to the fourth embodiment will be described. The same components as those of the imaging apparatus 1 according to the above-described first embodiment are denoted by the same reference numerals and will not be described.

Configuration of Imaging Apparatus

FIG. 17 is a block diagram illustrating a functional configuration of an imaging apparatus according to the fourth embodiment of the present invention. An imaging apparatus 1b illustrated in FIG. 17 further includes an optical filter 28 in addition to the configuration of the imaging apparatus 1 according to the above-described first embodiment.

The optical filter 28 is disposed at the front of a filter array 23, and transmits light of a first wavelength band including the respective transmission spectrum maximum values of visible light filters R, visible light filters G, and visible light filters B, and of a second wavelength band including the transmission spectrum maximum value of invisible light filters IR.

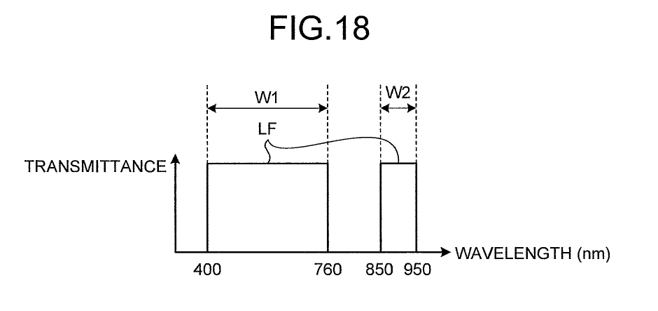

FIG. 18 is a graph illustrating the transmittance characteristics of the optical filter 28. In FIG. 18, the horizontal axis represents wavelength (nm), and the vertical axis represents transmittance. In FIG. 18, a broken line LF represents the transmittance characteristics of the optical filter 28.

As illustrated in FIG. 18, the optical filter 28 transmits light of a first wavelength band W1 including the respective transmission spectra of the visible light filters R, the visible light filters G, and the visible light filters B, and of a second wavelength band W2 of the transmission spectrum of the invisible light filters IR. Specifically, the optical filter 28 transmits light of 400 to 760 nm in a visible light range, and transmits light of 850 to 950 nm in an invisible light range. Thus, image data on visible light and image data on invisible light can be obtained separately. In FIG. 18, in order to simplify the description, the optical filter 28 transmits light of 400 to 760 nm in the visible light range, and transmits light of 850 to 950 nm in the invisible light range. As a matter of course, it may allow at least part of light having a wavelength band of 760 to 850 nm to pass through (not allow at least part of that to pass through). For example, the optical filter 28 may allow light having at least part of a wavelength band of 770 to 800 nm to pass through.

According to the fourth embodiment of the present invention described above, the optical filter 28 transmits light of the first wavelength band W1 including the respective transmission spectra of the visible light filters R, the visible light filters G, and the visible light filters B, and of the second wavelength band W2 including the transmission spectrum of the invisible light filters IR, thereby removing unnecessary information (wavelength components), so that an improvement in the precision of the visible light range can be realized (higher resolution), and the degree of freedom in an optical source used for the invisible light range can be improved. Image data for generating vital information on a subject can be obtained in a non-contact state.

### Fifth Embodiment

Next, a fifth embodiment of the present invention will be described. An imaging apparatus according to the fifth embodiment is different in configuration from the imaging apparatus 1 according to the above-described first embodiment. Specifically, the imaging apparatus according to the fifth embodiment further includes an irradiation unit that emits light of an invisible light range of wavelengths longer than those of a visible light range. Thus, hereinafter, a configuration of the imaging apparatus according to the fifth embodiment will be described. The same components as those of the imaging apparatus 1 according to the above-described first embodiment are denoted by the same reference numerals and will not be described.

Configuration of Imaging Apparatus

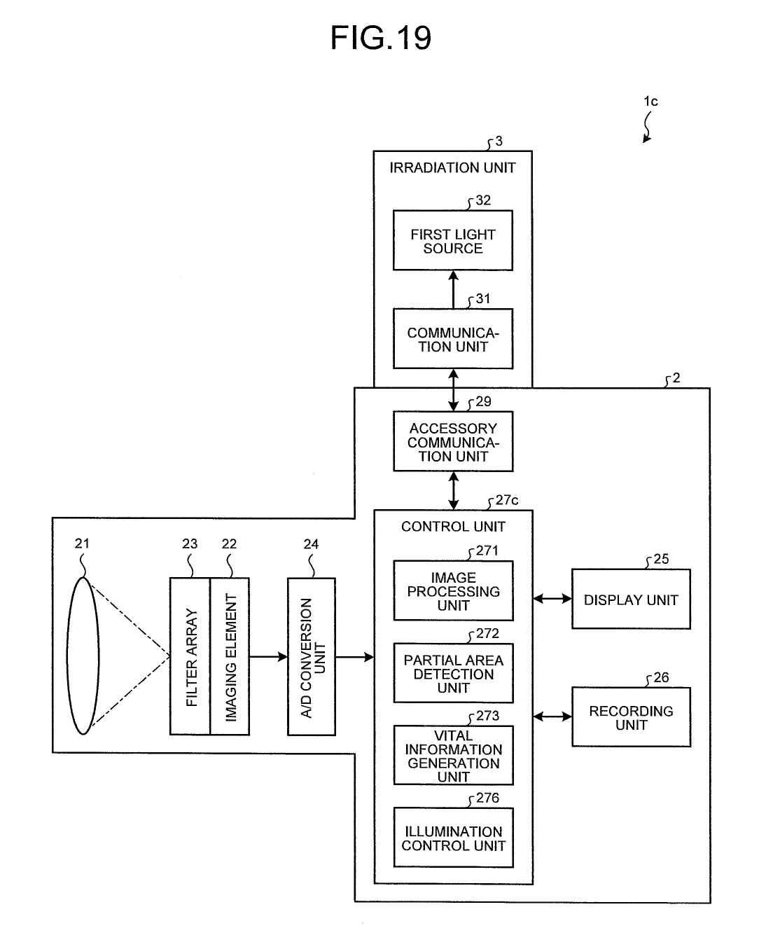

FIG. 19 is a block diagram illustrating a functional configuration of an imaging apparatus according to the fifth embodiment of the present invention. An imaging apparatus 1c illustrated in FIG. 19 includes a main body 2 that images a subject and generates image data on the subject, and an irradiation unit 3 that is detachably attached to the main body 2, and emits light having a predetermined wavelength band toward an imaging area of the imaging apparatus 1c.

Configuration of Main Body

First, a configuration of the main body 2 will be described.

The main body 2 includes an optical system 21, an imaging element 22, a filter array 23, an A/D conversion unit 24, a display unit 25, a recording unit 26, a control unit 27c, and an accessory communication unit 29.

The accessory communication unit 29 transmits a drive signal to an accessory connected to the main body 2, under the control of the control unit 27c, in compliance with a predetermined communication standard.

The control unit 27c performs instructions, data transfer, and so on to units constituting the imaging apparatus 1c, thereby centrally controlling the operation of the imaging apparatus 1c. The control unit 27c includes an image processing unit 271, a partial area detection unit 272, a vital information generation unit 273, and an illumination control unit 276.

The illumination control unit 276 controls light emission of the irradiation unit 3 connected to the main body 2 via the accessory communication unit 29. For example, in a case where a vital information generation mode to generate vital information on a subject is set in the imaging apparatus 1c, when the irradiation unit 3 is connected to the main body 2, the illumination control unit 276 causes the irradiation unit 3 to emit light in synchronization with imaging timing of the imaging element 22.

Configuration of Irradiation Unit

Next, a configuration of the irradiation unit 3 will be described. The irradiation unit 3 includes a communication unit 31 and a first light source 32.

The communication unit 31 outputs a drive signal input from the accessory communication unit 29 of the main body 2 to the first light source 32.

According to a drive signal input from the main body 2 via the communication unit 31, the first light source 32 emits, toward a subject, light having a wavelength band within a wavelength range that is transmitted by invisible light filters IR (hereinafter, referred to as “first wavelength light”). The first light source 32 is configured using a light emitting diode (LED).

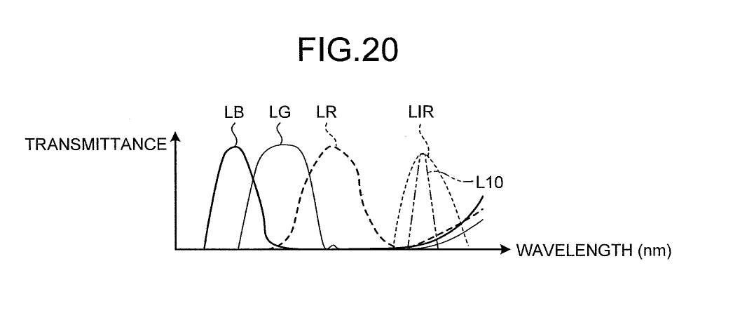

Next, the relationship between each filter and the first wavelength light emitted by the first light source 32 will be described. FIG. 20 is a graph illustrating the relationship between the transmittance characteristics of each filter and the first wavelength light emitted by the first light source 32. In FIG. 20, the horizontal axis represents wavelength (nm), and the vertical axis represents transmittance. In FIG. 20, a curved line LR represents the transmittance of visible light filters R, a curved line LG represents the transmittance of visible light filters G, a curved line LB represents the transmittance of visible light filters B, a curved line LIR represents the transmittance of the invisible light filters IR, and a curved line L10 represents a first wavelength band emitted by the first light source 32.

As illustrated in FIG. 20, the first light source 32 emits the first wavelength light having the wavelength band within the wavelength range transmitted by the invisible light filters IR, according to a drive signal input from the main body 2 via the communication unit 31. Specifically, the first light source 32 emits light of 860 to 900 nm.

According to the fifth embodiment of the present invention described above, the first light source 32 emits the first wavelength light that is within a second wavelength band W2 in an optical filter 28 and has a half-value width, a width less than or equal to half of the second wavelength band W2, so that image data for generating vital information on a subject can be obtained in a non-contact state.

Further, according to the fifth embodiment of the present invention, high-accuracy invisible light information can be obtained because the first wavelength light having the wavelength band within the wavelength range transmitted by the invisible light filters IR is emitted.

Although the first light source 32 emits light of 860 to 900 nm as the first wavelength light in the fifth embodiment of the present invention, it may be configured using an LED capable of emitting light of 970 nm when skin moisture is detected as vital information on a living body, for example. At this time, the optical filter 28 capable of transmitting light of an invisible light band of 900 to 1000 nm as the second wavelength band may be used.

In the fifth embodiment of the present invention, the vital information generation unit 273 may detect skin color variability of a subject, based on IR data from IR pixels in image data of the imaging element 22 input continuously from the A/D conversion unit 24 (hereinafter, referred to as “moving image data”), detect a heart rate/heart rate variability of the subject, based on respective RGB data of R pixels, G pixels, and B pixels in the moving image data, and detect an accurate heart rate of the subject, based on the detected heart rate/heart rate variability and the above-described skin color variability of the subject. Further, the vital information generation unit 273 may detect the degree of stress of the subject from a waveform of the above-described heart rate variability, as vital information.

Although the irradiation unit 3 is detachably attached to the main body 2 in the fifth embodiment of the present invention, the irradiation unit 3 and the main body 2 may be formed integrally.

### Sixth Embodiment

Next, a sixth embodiment of the present invention will be described. An imaging apparatus according to the sixth embodiment is different in configuration from the imaging apparatus 1c according to the above-described fifth embodiment. Thus, hereinafter, a configuration of the imaging apparatus according to the sixth embodiment will be described. The same components as those of the imaging apparatus 1c according to the above-described fifth embodiment are denoted by the same reference numerals and will not be described.

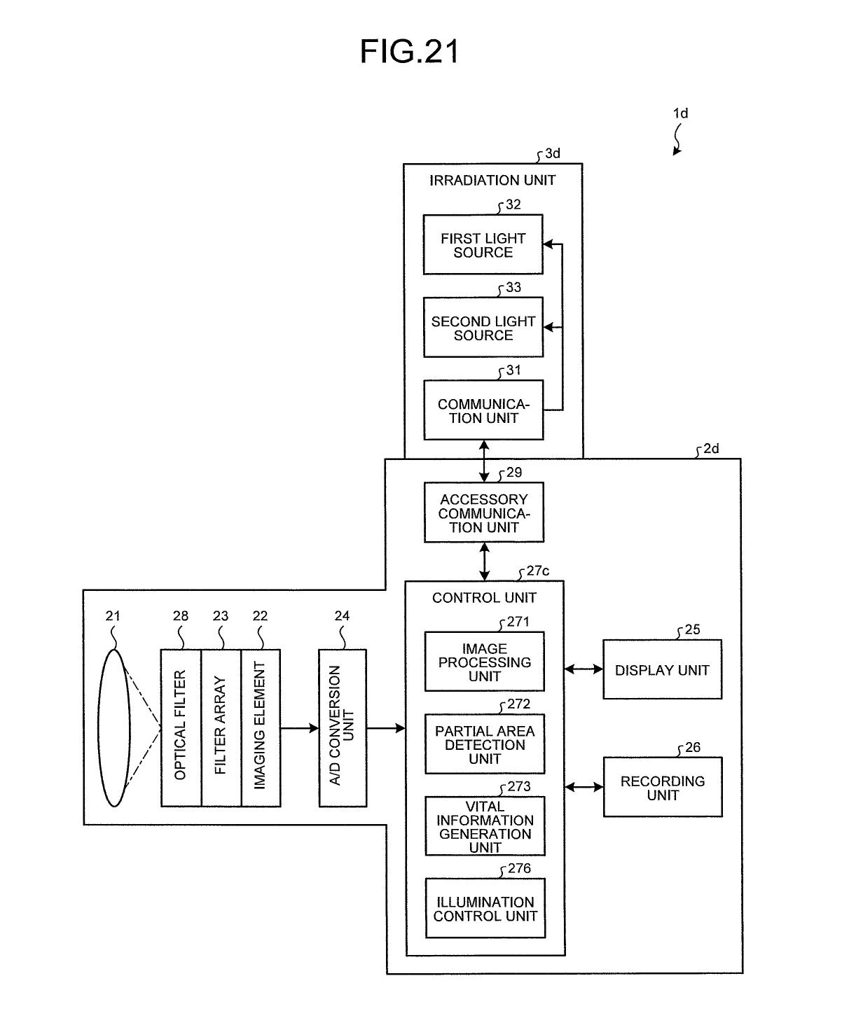

FIG. 21 is a block diagram illustrating a functional configuration of an imaging apparatus according to the sixth embodiment of the present invention. An imaging apparatus 1d illustrated in FIG. 21 includes a main body 2d and an irradiation unit 3d.

Configuration of Main Body

First, a configuration of the main body 2d will be described. The main body 2d further includes the optical filter 28 according to the above-described fourth embodiment in addition to the configuration of the main body 2 of the imaging apparatus 1c according to the above-described fifth embodiment.

Configuration of Irradiation Unit

Next, a configuration of the irradiation unit 3d will be described. The irradiation unit 3d emits light having a predetermined wavelength band toward an imaging area of the imaging apparatus 1d. The irradiation unit 3d further includes a second light source 33 in addition to the configuration of the irradiation unit 3 according to the above-described fifth embodiment.

The second light source 33 emits, toward a subject, light within the second wavelength band in the optical filter 28, light of a second wavelength that has a half-value width, a width less than or equal to half of the second wavelength band, and is different from light of the first wavelength. The second light source 33 is configured using an LED.

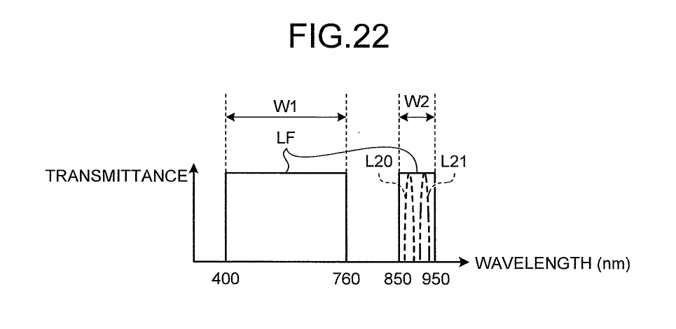

Next, the relationship between the above-described optical filter 28 and light of a first wavelength band emitted by the first light source 32 and light of a second wavelength band emitted by the second light source 33 will be described. FIG. 22 is a graph illustrating the relationship between the transmittance characteristics of the optical filter 28 and light of the first wavelength band emitted by the first light source 32 and light of the second wavelength band emitted by the second light source 33. In FIG. 22, the horizontal axis represents wavelength (nm), and the vertical axis represents transmittance. In FIG. 22, a broken line LF represents the transmittance characteristics of the optical filter 28, a curved line L20 represents the wavelength band of light emitted by the first light source 32, and a curved line L21 represents the wavelength band of light emitted by the second light source 33.

As illustrated in FIG. 22, the optical filter 28 only transmits respective light of a first wavelength band W1 of visible light filters R, visible light filters G, and visible light filters B, and light of a second wavelength band W2 of invisible light filters IR. As shown by the curved line L20, the first light source 32 emits light of the first wavelength band W1 that is within the second wavelength band W2 transmitted by the optical filter 28 and has a half-value width, a width less than or equal to half of the second wavelength band. Further, as shown by the curved line L21, the second light source 33 emits light of the second wavelength band that is within the second wavelength band W2 transmitted by the optical filter 28 and has a half-value width less than or equal to half of the second wavelength band W2. Further, the second light source 33 emits light of the second wavelength band W2 having a wavelength band different from light of the first wavelength band emitted by the first light source 32. Specifically, the second light source 33 emits light of 940 to 1000 nm.

By the illumination control unit 276 causing the first light source 32 and the second light source 33 to emit light alternately, the imaging apparatus 1d configured like this can obtain vital information, and also can obtain space information and distance information on a three-dimensional map produced by 3D pattern projection.

According to the sixth embodiment of the present invention described above, the second light source 33 that emits, toward a subject, light within the second wavelength band in the optical filter 28, light of the second wavelength that has a half-value width less than or equal to half of the second wavelength band, and is different from light of the first wavelength is further provided, and the illumination control unit 276 causes the first light source 32 and the second light source 33 to emit light alternately, so that vital information can be obtained, and also space information and distance information on a three-dimensional map produced by 3D pattern projection can be obtained.

Further, according to the sixth embodiment of the present invention, the first light source 32 and the second light source 33 may emit different near-infrared light (e.g. 940 nm and 1000 nm), and the vital information generation unit 273 may generate oxygen saturation in a skin surface as vital information, based on IR data on a partial area.

Although the illumination control unit 276 causes the first light source 32 and the second light source 33 to emit light alternately in the sixth embodiment of the present invention, light emission timings may be changed at intervals of a predetermined number of frames of image data generated by the imaging element 22, for example. Further, the illumination control unit 276 may switch between the first light source 32 and the second light source 33 according to the respective numbers of light emissions.

### Other Embodiments

Although in the above-described fifth and sixth embodiments, the first light source or the second light source is configured using an LED, it may alternatively be configured using a light source that emits light of a visible light wavelength band and a near-infrared wavelength band like a halogen light source, for example.

Although in the above-described first to sixth embodiments, as visible light filters, primary color filters, the visible light filters R, the visible light filters G, and the visible light filters B, are used, complementary color filters such as magenta, cyan, and yellow, for example, may alternatively be used.

Although in the above-described first to sixth embodiments, the optical system, the optical filter, the filter array, and the imaging element are built into the main body, the optical system, the optical filter, the filter array, and the imaging element may alternatively be housed in a unit, and the unit may be detachably attached to the image processing apparatus as a main body. As a matter of course, the optical system may be housed in a lens barrel, and the lens barrel may be configured to be detachably attached to a unit housing the optical filter, the filter array, and the imaging element.

In the above-described first to sixth embodiments, the vital information generation unit is provided in the main body. Alternatively, for example, a function capable of generating vital information may be actualized by a program or application software in a mobile device or a wearable device such as a watch or glasses capable of bidirectional communication, and by transmitting image data generated by an imaging apparatus, the mobile device or the wearable device may generate vital information on a subject.

The present invention is not limited to the above-described embodiments, and various modifications and applications may be made within the gist of the present invention, as a matter of course. For example, other than the imaging apparatus used to describe the present invention, the present invention can be applied to any apparatus capable of imaging a subject, such as a mobile device or a wearable device equipped with an imaging element in a mobile phone or a smartphone, or an imaging apparatus for imaging a subject through an optical device, such as a video camera, an endoscope, a surveillance camera, or a microscope.

A method of each processing by the image processing apparatus in the above-described embodiments, that is, any processing illustrated in the timing charts may be stored as a program that the control unit such as a CPU can be caused to execute. Besides, it can be stored in a storage medium of an external storage device such as a memory card (such as a ROM card or a RAM card), a magnetic disk, an optical disk (such as a CD-ROM or a DVD), or semiconductor memory for distribution. The control unit such as a CPU reads a program stored in the storage medium of the external storage device, and by the operation being controlled by the read program, the above-described processing can be executed.

According to the disclosure, it is possible to obtain vital information on a living body in a non-contact state.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An imaging apparatus that generates image data for detecting vital information on a subject, the imaging apparatus comprising:
an imaging element that generates the image data by photoelectrically converting light received by each of a plurality of pixels arranged two-dimensionally;
a filter array including a unit including different types of visible light filters with different transmission spectrum maximum values within a visible light band, and invisible light filters having a transmission spectrum maximum value in an invisible light range of wavelengths longer than those of the visible light band, the visible light filters and the invisible light filters being disposed in correspondence with the plurality of pixels;
a partial area detection unit that detects a partial area of the subject on an image corresponding to the image data generated by the imaging element; and
a vital information generation unit that generates vital information on the subject, based on image signals output by pixels, in an imaging area of the imaging element corresponding to the partial area detected by the partial area detection unit, on which the invisible light filters are disposed.

2. The imaging apparatus according to claim 1, wherein number of the invisible light filters is lower than number of the visible light filters of each type.

3. The imaging apparatus according to claim 1, further comprising an optical filter disposed on a light-receiving surface of the filter array, the optical filter transmitting light included in either a first wavelength band that includes the respective transmission spectrum maximum values of the different types of visible light filters or a second wavelength band that includes the transmission spectrum maximum value of the invisible light filters.

4. The imaging apparatus according to claim 3, further comprising a first light source that emits, toward the subject, light having a wavelength within the second wavelength band, light of a first wavelength having a half-value width less than or equal to half of the second wavelength band.

5. The imaging apparatus according to claim 1, further comprising a first light source that emits, toward the subject, light having a wavelength in a transmission wavelength band of the invisible light filters.

6. The imaging apparatus according to claim 1, wherein the vital information generation unit generates vital information on the subject, based on image signals output by pixels on which the different types of visible light filters are disposed and image signals output by pixels on which the invisible light filters are disposed, of pixels in an imaging area of the imaging element corresponding to the partial area detected by the partial area detection unit.

7. The imaging apparatus according to claim 1, wherein when the partial area detection unit detects a plurality of the partial areas, the vital information generation unit generates vital information on the subject on each of the plurality of partial areas.

8. The imaging apparatus according to claim 1, wherein the vital information generation unit divides the partial area detected by the partial area detection unit into a plurality of areas, and generates vital information on the subject on each of the plurality of divided areas.

9. The imaging apparatus according to claim 1, further comprising:
a luminance determination unit that determines whether or not the luminance of an image corresponding to the image data generated by the imaging element is more than or equal to predetermined luminance, wherein
when the luminance determination unit determines that the luminance of the image is more than or equal to the predetermined luminance, the partial area detection unit detects the partial area based on image signals output by pixels on which the visible light filters are disposed, and on the other hand, when the luminance determination unit determines that the luminance of the image is not more than or equal to the predetermined luminance, the partial area detection unit detects the partial area based on image signals output by pixels on which the visible light filters are disposed and image signals output by pixels on which the invisible light filters are disposed.

10. The imaging apparatus according to claim 1, wherein
the imaging element continuously generates the image data,
the partial area detection unit sequentially detects the partial area on an image corresponding to the image data continuously generated by the imaging element, and
the vital information generation unit generates the vital information every time the partial area detection unit detects the partial area.

11. The imaging apparatus according to claim 1, wherein the vital information is at least one of blood pressure, a heart rate, heart rate variability, stress, oxygen saturation, skin moisture, and a vein pattern.

12. An image processing apparatus that generates vital information on a subject using image data generated by an imaging apparatus that comprises an imaging element that generates the image data by photoelectrically converting light received by each of a plurality of pixels arranged two-dimensionally, and a filter array having a unit including different types of visible light filters with different transmission spectrum maximum values within a visible light band, and invisible light filters having a transmission spectrum maximum value in an invisible light range of wavelengths longer than those of the visible light band, disposed in correspondence with the plurality of pixels, the image processing apparatus comprising:
a partial area detection unit that detects a partial area of the subject on an image corresponding to the image data; and
a vital information generation unit that generates vital information on the subject, based on image signals output by pixels, in an imaging area of the imaging element corresponding to the partial area detected by the partial area detection unit, on which the invisible light filters are disposed.

13. An image processing method executed by an image processing apparatus that generates vital information on a subject using image data generated by an imaging apparatus that comprises an imaging element that generates the image data by photoelectrically converting light received by each of a plurality of pixels arranged two-dimensionally, and a filter array including a unit including different types of visible light filters with different transmission spectrum maximum values within a visible light band, and invisible light filters having a transmission spectrum maximum value in an invisible light range of wavelengths longer than those of the visible light band, disposed in correspondence with the plurality of pixels, the image processing method comprising:
detecting a partial area of the subject on an image corresponding to the image data; and
generating vital information on the subject based on image signals output by pixels, in an imaging area of the imaging element corresponding to the detected partial area, on which the invisible light filters are disposed.
